(19) United States
(12) Patent Application Publication
(10) Pub. No.: US 2023/0343421 A1
(43) Pub. Date: Oct. 26, 2023

(54) CENTRALIZED PLATFORM FOR MANAGEMENT OF SUBSTANCE ABUSE TREATMENT PROGRAMS

(71) Applicant: Kyros Care PBC, Minneapolis, MN (US)

(72) Inventors: DANIEL LARSON, Minneapolis, MN (US); JOHN MIKKELSEN, Minnetonka, MN (US); SCOTT BISHOP, Minneapolis, MN (US); MARC SEABERG, Edina, MN (US)

(21) Appl. No.: 18/306,417

(22) Filed: Apr. 25, 2023

Related U.S. Application Data
(60) Provisional application No. 63/334,457, filed on Apr. 25, 2022.

(51) Int. Cl. G16H 10/60; G16H 40/20

(52) U.S. Cl. CPC G16H 10/60; G16H 40/20

(57) ABSTRACT

A centralized platform for management of substance abuse treatment programs is disclosed. The platform tracks recovery user status across a plurality of third party entities, including treatment entities, healthcare entities, government-mandated programs, and employment-related entities. The user and one or more third party entities may be provided with a set of user interfaces for tracking progress through various treatment phases, and presented with verifiable certification of completion that is coordinated across the third party entities. In some example aspects, the recovery user may be qualified, upon completion of predetermined recovery and treatment program criteria and other training criteria, as a peer recovery specialist. In such instances, the centralized platform may enable the recovery user to be presented as a contact for other, subsequent recovery users to work with in completing various recovery programs.

### CROSS-REFERENCE TO RELATED APPLICATIONS

The present application claims priority from U.S. Provisional Patent Application No. 63/334,457, filed on Apr. 25, 2022, the disclosure of which is hereby incorporated by reference in its entirety.

### BACKGROUND

There are approximately 18,000 Substance Use Disorder (SUD) “Sober Living Houses” in the United States. A Sober Living House is a living facility with a certain number of beds allowing someone recently coming out of SUD treatment to transition back to a life free of addiction. More specifically a Sober Living House provides safe housing and supportive, structured living conditions for people exiting SUD rehabilitation programs. Sober Living Housing serves as a transitional environment between such programs and mainstream society.

Each Sober Living House organization has at least one “house manager,” who manages a given Sober Living House location(s). Each Sober Living House also houses anywhere from approximately 10 to 100 SUD transitioning individuals.

A training entity will train any and all willing “Sober House Managers” interested in becoming a certified Peer Recovery Specialist (PRS) of any Sober Living House. Certification, as well as testing, is handled through a state board of the state in which the Sober Living House is located. There is no national test.

Additionally, people suffering from Substance Use Disorder (SUDs) have a hard time accessing resources and receiving support from services that are offered by the state in which they reside.

In addition, tracking of information in this environment is also difficult, and non-standardized. For example, Treatment Centers chart for billing purposes, not for collecting data around the service that a client receives. Most use a variety of different systems and planning methodologies that makes it difficult to compare approaches. As such, the information collected from each of these entities is largely not comparable, as it includes different collections of information, in different formats. As a still further example, insurance companies issuing state sponsored healthcare plans are left being reactive by the lack of historical data and intangible efforts (non-billable treatment events) that a person goes through when they have finally had enough of their addiction.

There is no central database for all the time and effort that someone puts into their recovery and many times treatment services are missed or unrecorded. There is also no place that consolidates resources, documents progress and records time investments while communicating success simultaneously. Today's systems are all organization and role specific; they do not focus on the client or assess any metrics of success beyond the person entering the data.

It is in this environment in which aspects of the present system are implemented.

### SUMMARY

In example implementations, a centralized platform for management of substance abuse treatment programs is provided that tracks recovery user status across a plurality of third party entities, including treatment entities, government-mandated programs, and employment-related entities. The user may be provided with a set of user interfaces for tracking progress through various treatment phases and presented with verifiable certification of completion that is coordinated across the third party entities. In some example aspects, the recovery user may be qualified, upon completion of predetermined recovery and treatment program criteria and other training criteria, as a peer recovery specialist. In such instances, the centralized platform may enable the recovery user to be presented as a contact for other, subsequent recovery users to work with in completing various recovery programs.

In an example aspect, a platform for management of substance abuse treatment programs implemented on a computing system is disclosed. The platform includes a centralized datastore managing a plurality of recovery users, the datastore storing, for each recovery user, the recovery user's demographic information, personal preferences, a treatment status, peer recovery certifications, related recovery users, and a transaction history. The platform further includes a plurality of data interfaces accessible by and from third party systems including medical and counseling centers, government officers, and third party entities authorized by a recovery user, the plurality of data interfaces being configured to receive treatment and employment records from the third party systems. The platform also includes a plurality of user interfaces presentable to the recovery user, the plurality of user interfaces including one or more user certification interfaces presenting a treatment status of the recovery user.

In a further aspect, a method of managing substance abuse recovery process for a recovery user at a centralized recovery platform is disclosed. The method includes receiving a registration of a recovery user in a program managed using the centralized recovery platform and establishing a relationship with a peer recovery specialist via the centralized recovery platform. The method further includes tracking progress of the recovery user through treatment programs and concurrently monitoring status of the recovery user in one or more third party monitoring systems, the status including at least one of government status and employment status, and, during the progress of the recovery user through the treatment programs, providing the recovery user with information regarding qualification as a peer recovery specialist. The method also includes, upon meeting predetermined recovery criteria and training criteria, qualifying the recovery user as a peer recovery specialist at the centralized recovery platform.

### BRIEF DESCRIPTION OF THE DRAWINGS

FIG. 1 illustrates an example environment in which aspects of the present disclosure can be implemented.

FIG. 2 illustrates an example computing system on which aspects of the present disclosure can be implemented.

FIG. 3 is an example schematic diagram of logical utilization of a centralized platform according to aspects of the present disclosure.

FIG. 4 is an example of a user interface depicting a recovery user account and status page managed using the centralized platform of the present disclosure.

FIG. 5 is an example of a recovery credibility report user interface generated using the centralized platform of the present disclosure.

FIG. 6 is an example of a user interface for meeting logs and meeting attendance assistance able to be monitored and implemented using the centralized platform of the present disclosure.

FIG. 7 is an example method of managing records and personnel across substance abuse treatment programs may be implemented.

FIG. 8 is an example system flow within the centralized platform for identifying and managing peer recovery specialist match recommendations, in an example embodiment.

FIG. 9 is an example system flow within the centralized platform for predictive behavior identification and notification processing, in an example embodiment.

### DETAILED DESCRIPTION

As briefly described above, embodiments of the present invention are directed to a centralized platform on which individuals may interact within a recovery community, as well as ways in which others outside the recovery community may interact with those within it. In example implementations, the platform provides controlled access and communication pathways via social media platforms, while providing recovery services and staffing processes as well as progress monitoring for recovering individuals.

In example embodiments, the methods and systems described herein provide centralized management of “Certified PRS Managers” to specifically represent the “SUD Clients” who may be residing in each given Sober House (within which the same managers, now certified PRS Managers, reside and/or work) or elsewhere. This improves and standardizes training for house managers to allow de-escalation techniques and provide a client-centered model. The result of this should improve SUD outcomes by a significant percentage over standard PRS/SUD client relationships.

This business methodology and process gives both the SUD Client (needing specific, necessary, and certified help), and the peer recovery specialist manager (PRS Manager) (providing the specific, necessary, and certified help) an “already familiar relationship” from which to build upon, as the PRS Manager and the SUD Client already reside together and/or work together in the same Sober House. This potentially proprietary method of activity and business can result in a better, more fulfilling relationship (and outcome for the SUD Client), as there is already a foundational relationship with the PRS/Manager from which to build upon.

This narrow and specific method of business, within the massive Substance Use Disorder industry, can be duplicated with Managers and SUD Clients residing/working together within any Sober House in the country. This can be accomplished via platform/application, and/or a simple SAAS model.

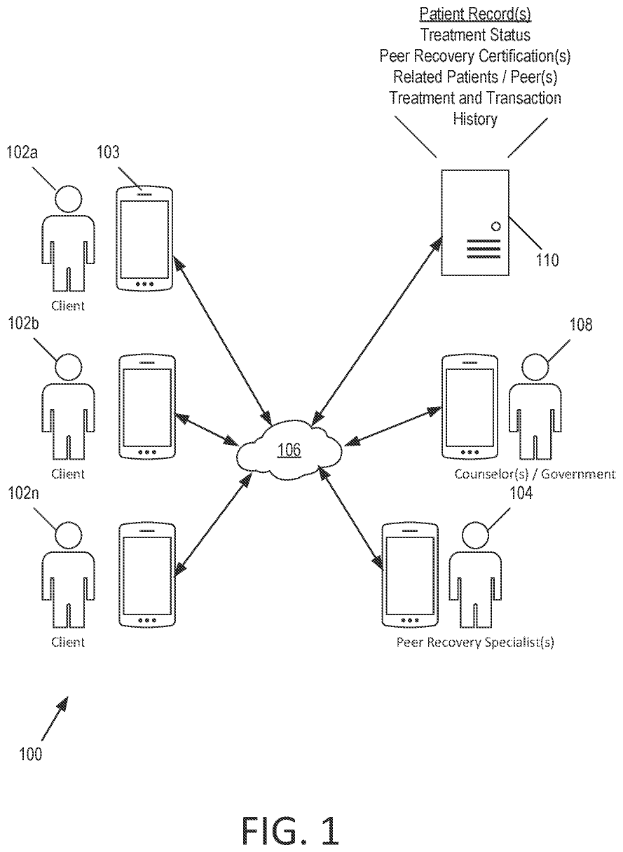

Referring to FIG. 1, an example environment in which aspects of the present innovation may be implemented is shown. The environment 100 illustrates a number of SUD Clients, referred to as Clients 102a-n (also referred to herein as “recovery users”) having computing devices 103 (such as mobile devices as shown) which may be communicatively connected to a centralized platform 110 via a network 106. Additionally, a peer recovery specialist 106 is similarly shown as having a computing device communicatively connected to the centralized platform 110, e.g., via network. The network 106 can be any of a variety of types of public and/or private networks, such as the Internet.

The Clients 102a-n may each be individuals at varying stages within a recovery process, and may be located across the country (e.g., across different states). Additionally, the peer recovery specialist 106 may be located anywhere within the country, either near or remote from Clients 102a-n and/or the centralized platform 110.

In example implementations, each client 102 will access the centralized platform 110 to view user interfaces, for example tracking patient records associated with each client. The patient records may include, for example, a treatment status of that recovery user (client), any peer recovery certifications held by that client, any related patients or peer recovery specialists associated with the client, as well as a treatment and transaction history of the client. The clients 102 may access the centralized platform 110 via user devices 103, for example via a network 106 such as the Internet.

In addition to clients 102, a variety of other entities may interact with the centralized platform 110. In some examples, such as the one shown here, a peer recovery specialist 104 may access records or be registered on the centralized platform 110, for example to provide various counseling and recovery services for the client. For example, the peer recovery specialist 104 may perform various paid tasks for the client/recovery user, such as driving the client to parole officer appointments, taking them to doctors appointments, buying groceries and the like. The peer recovery specialist 104 may also be paid to chaperone the Client 102 to entertainment activities such as sporting events, concerts, movies, parks, museums and the like.

In example implementations the centralized platform 110 may receive information from, and provide information to, various third party services or service providers via data interfaces. For example, the centralized platform 110 may interact with counselors, government entities, employers, and the like (generally, third party entities 108) to receive records of completion of various mandated programming, current employment status, completion of counseling processes, and the like. Such information may be gathered within the patient record at the centralized platform 110 and may be combined at that platform to provide a holistic look at recovery of the client across all entities. In example embodiments, the centralized platform 110 has one or more additional external interfaces configured for communicative connection to partner entities, including referral entities and governmental resources. Referral entities may include, for example, insurance programs and third party counseling services that may offer services to the client 102.

In example embodiments, social media integration may be provided among clients 102a-n and/or peer recovery specialists 104, can allow those individuals in recovery to connect with others who may also be in recovery or may be in counseling roles, and establish a ready-made, turnkey network of support. The recovery services and staffing process enables users to request on demand support services to gain access to resources that the individual may require in at least near real time.

The progress monitoring processes provided by the platform allow a client 102 to view a real-time dashboard of that user's progress, as well as actionable suggestions on next steps and a comprehensive report that the user can provide in situations where instant credibility is needed (e.g. for purposes of obtaining housing, employment, and the like).

In example implementations, a person in recovery, also referred to herein as a substance abuse disorder client (SUD client) may be in current treatment or may have completed treatment. Such an individual may be connected with a peer recovery specialist by the integrated platform to ensure an efficient transition from treatment to post-treatment life for the SUD client. The client 102 may be supported by the peer recovery specialist 104, who is, during counseling and support, trained as a peer recovery specialist. The client 102 may then be able to register as a peer recovery specialist 104 and offer services to other clients in the future.

In some examples, each client who becomes a peer recovery specialist may be able to provide support and counseling for between one to ten other clients. Each of those clients may have progress tracked within the platform as well, and as they continue to develop in their own treatment program, each of those clients may be presented with the option of becoming a peer recovery specialist. Based on adoption rate, clients may provide a source of subsequent peer recovery specialists. In some examples, the centralized platform manages incentives for clients to become and maintain training/certification as peer recovery specialists, including providing additional platform features, incentive payments, and the like.

In example embodiments, the platform automatically identifies opportunities for particular openings to SUD clients to become peer recovery specialists, as well as presenting goals and measurable benchmarks not only along a particular treatment and recovery timeline, but also for training toward certification as a peer recovery specialist. This allows the centralized platform 110 to act as a self-service interface for clients 102 to obtain counseling services, insurance or other financial assistance, view and register completion of government-mandated programming, and interact with peers and peer recovery specialists, while generating reports for the user to certify a particular completion status of treatment programs and/or counseling that may be required for certification as a peer recovery specialist, or may be required by law or court order.

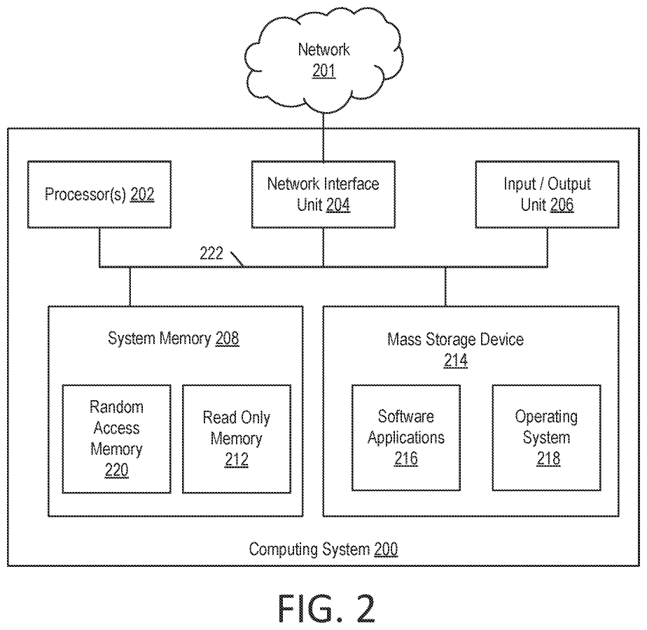

FIG. 2 illustrates an example block diagram of a virtual or physical computing system 200. One or more aspects of the computing system 200 can be used to implement the processes described herein, in particular, to operate as the centralized platform 110 or one of the devices 103 as described herein.

In the embodiment shown, the computing system 200 includes one or more processors 202, a system memory 208, and a system bus 222 that couples the system memory 208 to the one or more processors 202. The system memory 208 includes RAM (Random Access Memory) 210 and ROM (Read-Only Memory) 212. A basic input/output system that contains the basic routines that help to transfer information between elements within the computing system 200, such as during startup, is stored in the ROM 212. The computing system 200 further includes a mass storage device 214. The mass storage device 214 stores software instructions and data. The one or more processors 202 can be one or more central processing units or other processors.

The mass storage device 214 is connected to the one or more processors 202 through a mass storage controller (not shown) connected to the system bus 222. The mass storage device 214 and its associated computer-readable data storage media provide non-volatile, non-transitory storage for the computing system 200. Although the description of computer-readable data storage media contained herein refers to a mass storage device, such as a hard disk or solid state disk, it should be appreciated by those skilled in the art that computer-readable data storage media can be any available non-transitory, physical device or article of manufacture from which the central display station can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, DVD (Digital Versatile Discs), other optical storage media, magnetic cassettes, magnetic tape, magnetic disk storage or other magnetic storage devices, or any other medium which can be used to store the desired information and which can be accessed by the computing system 200.

According to various embodiments of the invention, the computing system 200 may operate in a networked environment using logical connections to remote network devices through the network 201. The network 201 is a computer network, such as an enterprise intranet and/or the Internet. The network 201 can include a LAN, a Wide Area Network (WAN), the Internet, wireless transmission mediums, wired transmission mediums, other networks, and combinations thereof. The computing system 200 may connect to the network 201 through a network interface unit 204 connected to the system bus 222. It should be appreciated that the network interface unit 204 may also be utilized to connect to other types of networks and remote computing systems. The computing system 200 also includes an input/output controller 206 for receiving and processing input from a number of other devices, including a touch user interface display screen, or another type of input device. Similarly, the input/output controller 206 may provide output to a touch user interface display screen or other type of output device.

As mentioned briefly above, the mass storage device 214 and the RAM 210 of the computing system 200 can store software instructions and data. The software instructions include an operating system 218 suitable for controlling the operation of the computing system 200. The mass storage device 214 and/or the RAM 210 also store software instructions, that when executed by the one or more processors 202, cause one or more of the systems, devices, or components described herein to provide functionality described herein. For example, the mass storage device 214 and/or the RAM 210 can store software instructions that, when executed by the one or more processors 202, cause the computing system 200 to perform the processes described herein.

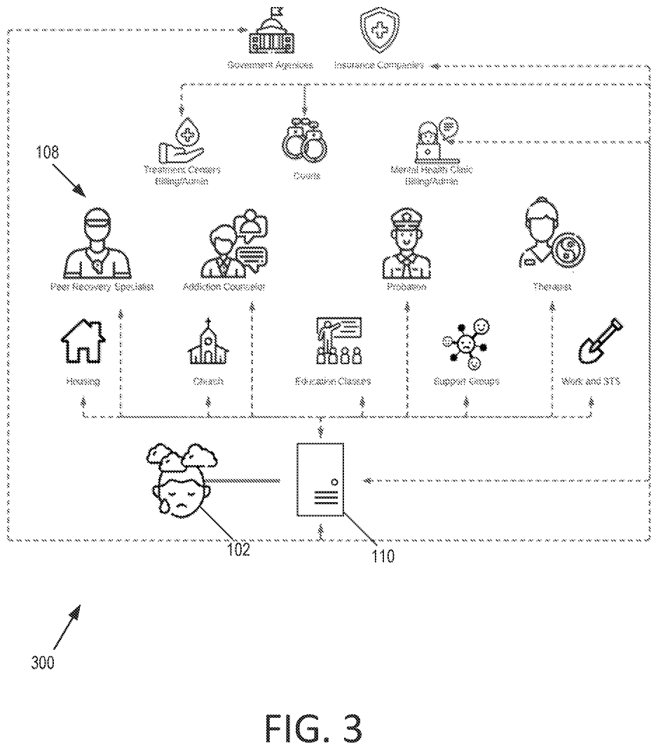

FIG. 3 is an example schematic diagram 300 of logical utilization of a centralized platform 110 according to aspects of the present disclosure. As shown, the client 102 may access the centralized platform 110 to view various data from, and support services from, recovery specialists, counselors, government officials such as probation officers, as well as therapists. Additionally, various information available to the client 102 may be provided, for example relating to area housing, church services, education classes, support groups, and various work opportunities. Such services may be hosted and presented to the user as ways to achieve required progress within a government or nongovernment certification program. Additionally, information from various treatment centers, courts, mental health clinics, insurance companies, and government agencies may be provided at the centralized platform 110, to present to the user various support services that may be available to them. Accordingly, the centralized platform 110 acts as a single point of contact for the client 102 and provides a convenient point of interaction for various third parties as well. Still further, the centralized platform 110 connects clients 102 to specific other clients, and peer recovery specialists, to assist with guiding them through the process of recovery.

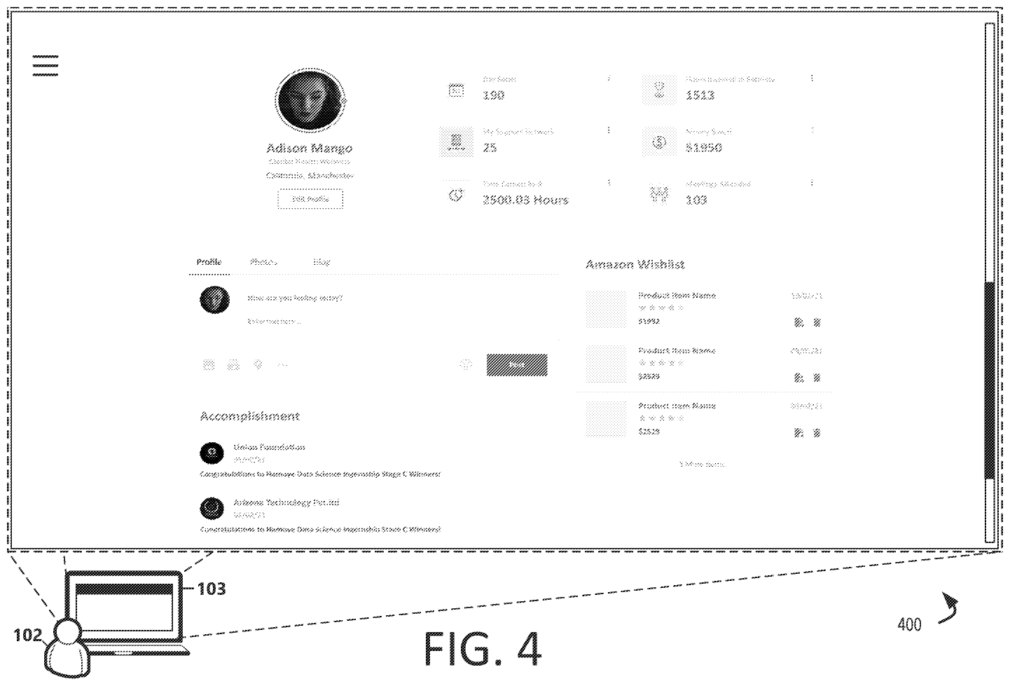

Referring now to FIGS. 4-6, various user interfaces presented by the centralized platform 110 are illustrated. The user interfaces may be presented to the client 102, at various stages within their recovery process. Referring first to FIG. 4, an example of a user interface 400 depicting a recovery user account and status page managed using the centralized platform of the present disclosure is depicted. The user interface 400 presents the user identity, as well as various status information related to that user. This can include, for example, an amount of time sober, number of meetings attended, money saved, and support network of social contacts within the centralized platform 110. The user interface 400 may be presented to the client 102 via the computing device 103 associated with that client.

In example implementations, the user interface 400 may present specific products and/or services to the user that may assist in the recovery process. The user interface 400 may also present specific accomplishments of the user or accomplishments of contacts of the user to facilitate interactivity among users of platform.

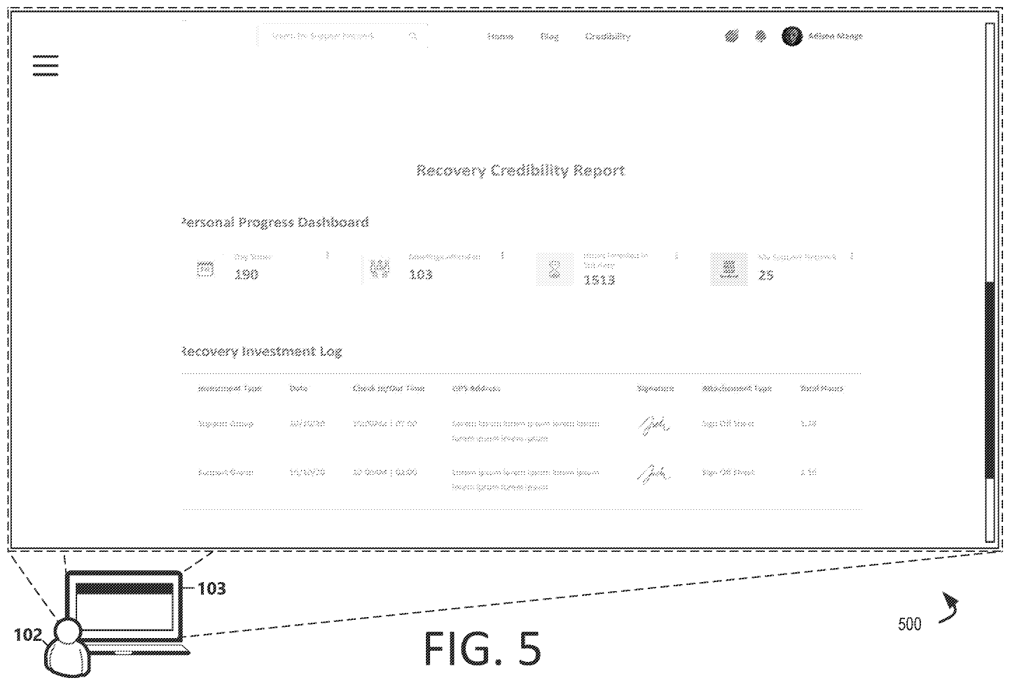

FIG. 5 is an example of a recovery credibility report user interface 500 generated using the centralized platform of the present disclosure. In the example shown, the recovery credibility report may be generated for an individual user and may present a personal progress of that user that is certified by receipt of information from various third parties, via interfaces with third-party platforms. In the example shown, the user interface 500 may present information about the user, such as number of days so per and the like, but may also present a recovery log indicating specific amounts of time spent in various recovery processes, such as counseling or group discussion. The recovery credibility report may include various other information that may be required to be presented by the user to third parties, for example to show continuous employment, continuous treatment, and the like for purposes of meeting government-imposed obligations.

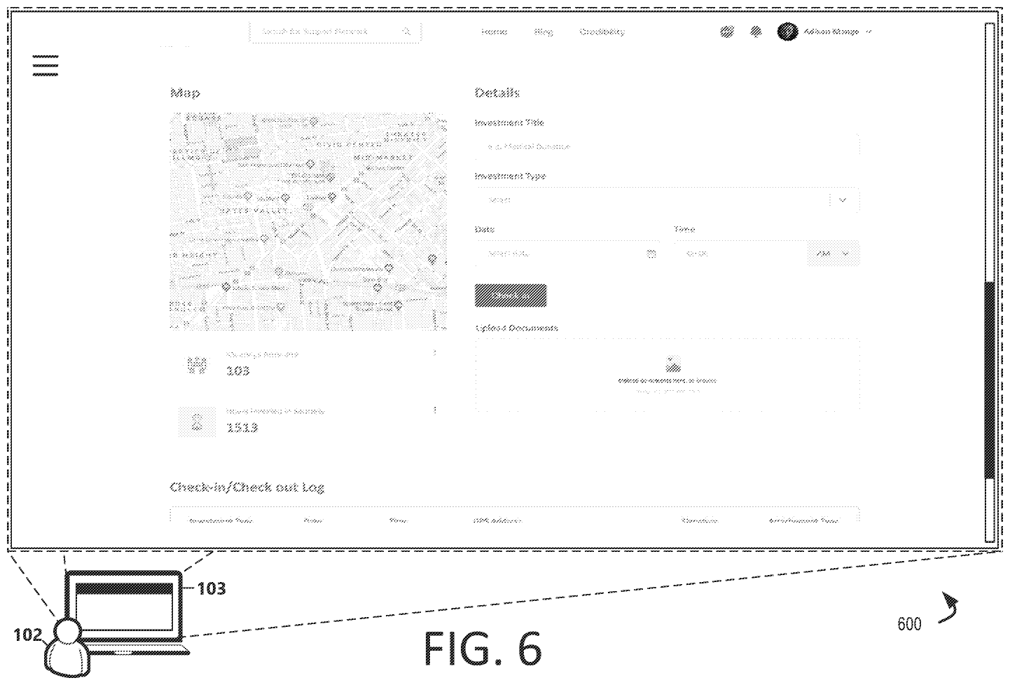

FIG. 6 is an example of a further user interface 600 for meeting logs and meeting attendance assistance able to be monitored and implemented using the centralized platform of the present disclosure. The user interface 600 presents to the client 102 an entry form that the user may utilize to assist that user in registering specific treatment meetings that the user has attended, tracking time in such meetings, and like. The user interface 600 may also be used to assist the client 102 in determining a location and obtaining a ride (if necessary) to the location of treatment.

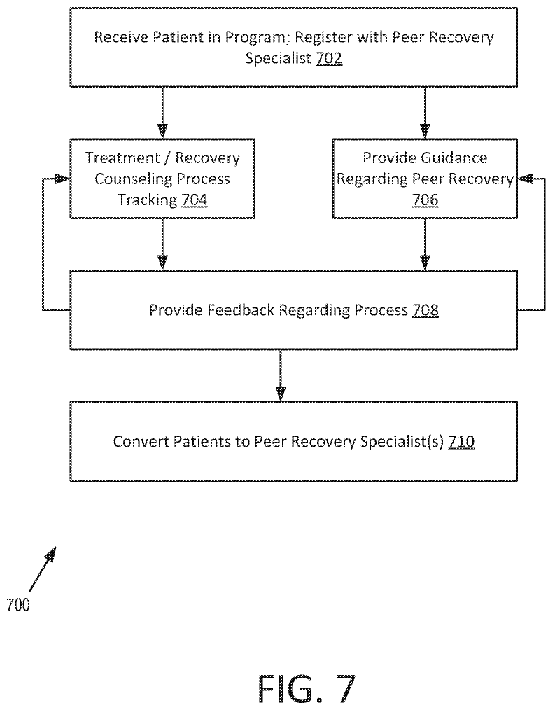

FIG. 7 is a depiction of a flowchart illustrating an example method 700 of managing records and personnel across substance abuse treatment programs may be implemented. The method 700 may be performed, for example, by the centralized platform 110, as well as computing systems associated with clients 102, peer recovery specialists 104, and various other third parties as depicted above in conjunction with FIGS. 1 and 3.

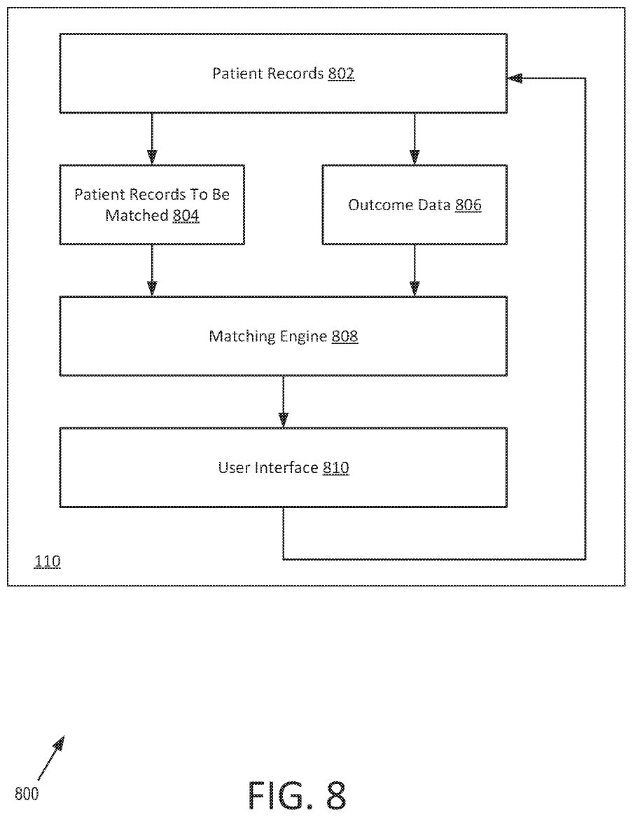

In the example shown, the method 700 includes receiving a registration of a patient, such as a recovery user, and a program managed using a centralized platform 110. Receiving the patient may occur at a predetermined time, e.g., while the patient is in treatment or exiting treatment. In such instances, the patient may be specifically referred to register with the centralized platform 110, for example by receiving a link or referral code to rapidly or conveniently register him/herself, as well as to associate his or her account with one or more third party treatment programs and/or governmental entities to which that user may be associated. The method may also include, registering that recovery user (e.g., client 102) at an ideal time in a centralized platform 110 with a peer recovery specialist (step 702). The peer recovery specialist may be made available via the platform 110, or may be assigned to the user based on geographic proximity, or various other matching processes. An example process for matching a recovery user with a peer recovery specialist is described below in conjunction with FIG. 8.

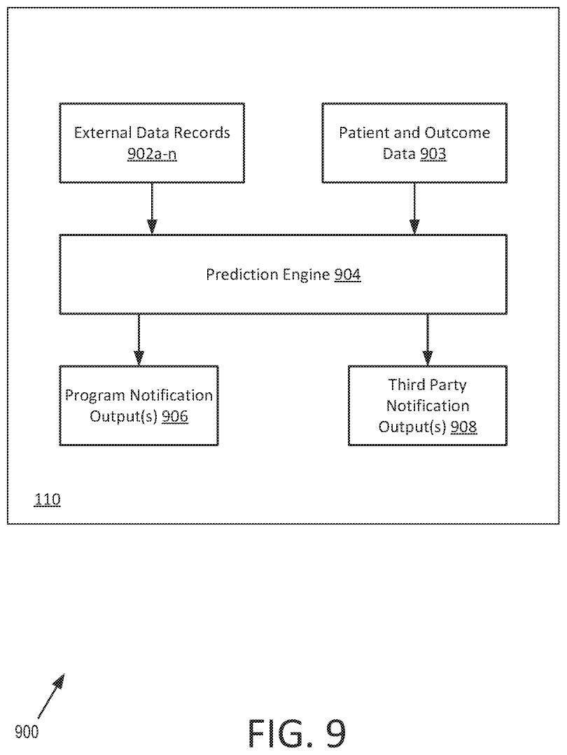

In the example shown, the method 700 further includes tracking various treatment and recovery counseling processes (step 704). This may include, for example, receiving from the user entry of information indicating completion of treatment programs, as well as receiving from third parties via data interfaces confirmation of completion of such recovery activities. In example embodiments, tracking treatment and recovery counseling processes for registered users may include executing one or more predictive algorithms and/or models usable to determine a likelihood of relapse based on observed behavior, whether self-reported, obtained from platform partners (e.g., healthcare entities and/or recovery personnel, law enforcement, and the like) or external data sources, such as obtainable information related to financial account transactions (shopping or spending patterns. Such tracking may include providing feedback to the user, for example in the form of extra guidance and/or intervention, as well as communication and/or notifications with one or more third parties (e.g., counselors, peer recovery specialists, healthcare and/or law enforcement personnel, and the like) in various forms. Details regarding predictive determinations and notifications are provided below in conjunction with FIG. 9.

Concurrently with tracking the various treatment and recovery counseling processes, the method 700 may also include providing guidance regarding qualification of the recovery user as a peer recovery specialist (step 706). The guidance regarding qualification of the recovery user as a peer recovery specialist may include presenting various benefits of becoming such a specialist, as well as presenting the required qualifications for becoming such a specialist, and opportunities for meeting those qualifications by meeting specific training, counseling, and other program criteria.

In the example shown, the method 700 also includes providing feedback regarding both the treatment and recovery counseling process, as well as progress regarding the process of qualification of the recovery user as a peer recovery specialist (step 708). This may be done through presentation, in various user interfaces (including, but not limited to, those presented above in conjunction with FIGS. 4-6).

In addition, the method 700 includes converting one or more of the recovery users, or patients, to peer recovery specialists, as those recovery users meet qualifications for that program (step 710). This may include a determination that the recovery user has met criteria, as well as receiving an indication from the user of interest in becoming such a peer recovery specialist. Upon receiving that confirmation and the completion of the criteria, the recovery user may in turn become available to be selected by other recovery users, or by the platform 110 itself, as a peer recovery specialist working with other recovery users in their general geographic area and meeting other matching criteria as may be applicable. The recovery user may be both a peer recovery specialist and maintain his or her own recovery process using the centralized platform 110, allowing that user to operate in various roles within the platform and with others in the recovery community.

Referring to FIG. 8, an example system flow within the centralized platform 110 is illustrated. The system flow illustrates processes and operational modules for identifying and managing peer recovery specialist match recommendations via a centralized platform using data insights obtained through recovery processes of a plurality of recovery users.

In the example shown, a set of patient records 802 may include records from both existing patients who are advanced within a recovery process, as well as new patients. New patients, also referred to as new recovery users, may need to be matched with peer recovery specialists. As discussed above, this matching may be based at least in part on recommendations generated by the platform 110. In the example shown, patient records for recovery users who do not have an associated peer recovery specialist may be extracted from the set of patient records overall. This set of to be matched patient records 804 may be provided to a matching engine 808, alongside outcome data 806. The outcome data 806 may be derived from patient records 802 of other recovery users, in particular those who have be monitored using the centralized platform 110 over time. Such outcome data 806 may include, for example, self-reported information regarding compatibility with peer recovery specialists, as well as inferential compatibility information.

Inferential compatibility information may correspond to various characteristics of peer recovery specialists and recovery users that may be correlated to recovery success via the matching engine 808. These may include, for example, common age range, common background, common gender, or other commonalities of the recovery user and to the peer recovery specialists that may assist the recovery user in communicating with the peer recovery specialist. However, these characteristics may also include characteristics of the peer recovery specialists that are not obvious or apparent correlated characteristics in common with the recovery user.

At the matching engine 808, outcome data 806 may be used to determine one or more recommendations of peer recovery specialists to present to a recovery user for selection, or otherwise to provide a matching service between recovery users and peer recovery specialists (e.g., in implementations where peer recovery specialists are automatically matched to users, rather than being presented via the centralized platform). The matching engine 808 may take a variety of forms. For example, in some implementations, the matching engine 808 may be implemented including a matching model that performs statistical correlations between characteristics of peer recovery specialists and outcomes of recovery users matched to those peer recovery specialists to identify characteristics of successful peer recovery specialists. The matching model may be implemented as a recommender model that determines correlations between characteristics of a given recovery user and a plurality of available peer recovery specialists, and correlates the closeness of characteristics between those two groups, alongside the success or failure rates of recovery by the recovery user in previously matched recovery user to peer recovery specialist pairs, to determine a correlation score. The correlation score may be generated for a new recovery user as to each of a plurality of potential candidate peer recovery specialists, which may then be rank ordered and presented to the user for selection via user interface 810. Example recommender models used within the matching engine 808 may include content-based filtering models and/or collaborative filtering models, deep learning models (e.g., convolutional neural networks and the like), or various hybrid models.

In some examples, the matching process performed by the matching engine 808 may be performed not just with respect to peer recovery specialists, but with respect to other services provided to the recovery user. That is, characteristics of social services programs, education classes, probation officers, work groups or particular employment, housing arrangements, and the like may be correlated to outcome data, and presented as recommendations to a recovery user within the centralized platform 110. In example implementations, separate machine learning models may be used to generate scores indicative of likely positive result for particular services, which may be rank ordered and presented to the user.

In some examples, recommended peer recovery specialists or other service providers may include only those available to the recovery user. However, in other examples, the recommended peer recovery specialists or other service providers may include all such service providers who may be a good match for the recovery user. In this instance, if the recovery user were to select a service or provider who is not available, a further matching process may be performed to provide a “best match” between an available service or specialist and the selected service or specialist from among the recommendations presented.

In the example implementations, matches selected by recovery users and/or peer recovery specialists via the user interface 810 may be linked to the patient record of the recovery user within the patient records 802. Accordingly, a feedback mechanism is provided by which the now-linked recovery user may be included among the patient records and outcome data analyze for other recovery users in the matching and recommendation process described herein.

Referring now to FIG. 9, a further example system flow within the centralized platform 110 is illustrated. The system flow illustrates processes and operational modules for predicting likelihood of substance abuse relapse by current users of the platform, as well as, in some instances, identifying potential non-users of the platform who may benefit from such a guided recovery system as described herein.

In the example shown, a set of external data records 902a-n may be ingested, alongside patient and outcome data 903, by a prediction engine 904. The external data records 902a-n (referred to generally as data records 902) may come from a plurality of different sources, such as electronic medical records (EMRs), or publicly available information, such as court records or other records generated by governmental entities. Additionally, such external data records 902 may include acquiring full data about both users and nonusers of the platform, such as credit reports, purchasing behavior, and the like. The patient and outcome data 903 may be specific to current or past users of the platform 110, and may include patient demographic data, including age, gender, occupation, and the like, as well as actions taken within the platform for purposes of recovery, other recovery efforts that are reported, and outcomes including both successful and unsuccessful (e.g., relapse) events.

The prediction engine 904 may include one or more predictive models usable to identify potential events for individual users or nonusers for which some type of communication or alerts may be beneficial. For a current user of the platform 110, the prediction engine 904 may be able to infer, based on a comparison of that individual's circumstances (e.g., demographic data, current behavior trends, and the like) to known outcomes for similarly situated individuals, that a probability of relapse may be elevated. For a non-user of the platform, one or more third party partners may provide some data to the platform regarding behavior of individuals that is derived from public records or private records (with consent) of the non-users. Such private records may include medical records, expense/financial or credit data, and the like. Based on these records, and the known outcome data available within the platform 110 in the form of patient and outcome data 903, predictions may be made regarding non-user likelihood of substance abuse and/or dependency.

The models included in the prediction engine 904 may include, for example, any of a variety of deep learning models, such as convolutional neural networks (CNN) or graph neural networks (GNN), Bayesian (e.g., statistical) models, regression models and/or decision tree models, and the like. In some examples, a plurality of different models are used with different combinations of received data.

In the example shown, the prediction engine 904 may generate both program notification output 906, and third party notification output 908. The program notification output 906 can take a number of forms. For example, for current users of the platform 110, program notification outputs 906 may include a communication sent to the user themselves, for example a check-in notification sent via email, text message, push notification, or the like. Such a notification may include a particular identification of potentially dangerous or relapse prone behavior taken by the individual, and suggestions of steps that may be taken to correct his or her actions. Additionally, notifications may be generated in the converse case as well, for example, to suggest productive behaviors and/or care plan steps that may be followed by the individual user based on a comparison to previously-successfully recovery users.

The program notification outputs 906 may also include notifications to other users of the platform 110, including peer recovery specialists, health caregivers, and the like. These notifications may include less detailed information, but may indicate a suggestion to check in with the platform user, to ensure his or her well-being, to recommend specific activities to do that may be productive, and the like. Additionally, for current platform users, third-party notification outputs 908 may include messages or alerts that can be ingested by third-party systems, such as healthcare records, insurance provider records, and the like. Such third-party notification outputs 908 may assist with identification of potential substance abuse issues to third parties to assist with non-platform treatment activities (e.g., in the case of third party healthcare or treatment).

For nonusers of the platform, there may be only a limited number of program notification outputs 906 that are available. In some instances, a non-user may know or be affiliated with a current user of the platform, in which case a platform user may receive a program notification output to contact the non-user. Because the program notification output is generated based on prediction, that output may merely recommend to the platform user that he or she discuss with the nonuser the benefits of the platform to the user, such that the nonuser may be enticed to begin treatment if in fact they have a substance abuse disorder. Third-party notification outputs 908, however, may take a variety of forms. This may include transmitting notifications to third-party providers, which may suggest a referral to the platform by, e.g., healthcare providers and the like. This may include targeted advertisements of the platform or messages suggesting enrollment in the platform to individuals or classes of individuals who meet criteria likely to involve substance abuse.

Referring to FIGS. 1-9 generally, it is noted that use of a centralized platform 110 has a number of advantages over existing approaches. For example, various advantages are achieved by integrated monitoring of recovery user status, including real-time in person, virtual, or phone crisis response; one-on-one mentoring by recovery specialists including accountability and goal setting; real-time printable creation of a credit report of that recovery user's investments in their own recovery to allow the user to bring proof of recovery progress to probation hearings, sentencing hearings, court appearances, child custody hearings, job interviews, housing applications and the like. Such a status may be maintained relative to specific recovery metrics or as a comparative over time. This information is verifiably maintained by a neutral third-party entity, and may be submitted to governmental entities, or other institutions where required. In existing solutions, such entities, such as courts, insurance companies, employers, rental agencies, and the like would have no specific insight into the progress a person is making and would have little ability to determine the likelihood of ultimate recovery for a given user. By presenting this information as a whole, such entities are capable of making better decisions with respect to the recovery user, leading to better outcomes.

Additionally, externally facing user interfaces that may be generated by the recovery platform may be made available externally to the recovery community, for example so the recovery user may publish his or her progress to friends, family, and the like so that they may offer encouragement.

While some example uses of the technology have been illustrated and discussed above, the disclosed technology can be used with a variety of data structures and processes in accordance with many examples of the technology. The above discussion is not meant to suggest that the disclosed technology is only suitable for implementation with the data structures shown and described above. For examples, while certain technologies described herein were primarily described in the context of a centralized platform for tracking and managing recovery from substance abuse disorders, technologies disclosed herein are applicable more generally to such systems.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

It should be appreciated, the various aspects (e.g., operations, memory arrangements, etc.) described with respect to the figures herein are not intended to limit the technology to the particular aspects described. Accordingly, additional configurations can be used to practice the technology herein and/or some aspects described can be excluded without departing from the methods and systems disclosed herein.

Similarly, where operations of a process are disclosed, those operations are described for purposes of illustrating the present technology and are not intended to limit the disclosure to a particular sequence of operations. For example, the operations can be performed in differing order, two or more operations can be performed concurrently, additional operations can be performed, and disclosed operations can be excluded without departing from the present disclosure. Further, each operation can be accomplished via one or more sub-operations. The disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

1. A platform for management of substance abuse treatment programs implemented on a computing system, the platform comprising:
a centralized datastore managing a plurality of recovery users, the datastore storing, for each recovery user, the user's demographic and personal information and preferences, a treatment status, peer recovery certifications, related recovery users, and a transaction history;
a plurality of data interfaces accessible by and from third party systems including medical and counseling centers, government officers, and third party entities authorized by a recovery user, the plurality of data interfaces being configured to receive treatment and employment records from the third party systems;
a plurality of user interfaces presentable to the recovery user, the plurality of user interfaces including one or more user certification interfaces presenting a treatment status of the recovery user.

2. The platform of claim 1, wherein the third party systems including medical and counseling centers, government officers, and third party entities authorized by a recovery user have the ability, through the platform, to correspond with one another more efficiently as it relates to a given client's case, based on the client's data-manifest being available for all parties to witness.

3. The platform of claim 1, wherein the platform implements a certification process for the recovery user to qualify that recovery user as a peer recovery specialist in response to meeting a predetermined set of treatment criteria.

4. The platform of claim 3, further comprising, upon the recovery user meeting the predetermined set of treatment criteria, enabling the recovery user to connect to other recovery users as a peer recovery specialist.

5. The platform of claim 1, wherein the plurality of user interfaces are presentable to a plurality of different third party users including a third party government user and a third party treatment provider, the plurality of different third party users being presented data regarding one or more associated recovery users.

6. The platform of claim 5, wherein the plurality of different third party users are enabled to communicate with each other regarding the associated recovery users.

7. The platform of claim 1, wherein the platform comprises a computing system configured to implement a recommendation workflow, the recommendation workflow being configured to generate recommendations regarding one or more peer recovery specialists and one or more service providers to the recovery user.

8. The platform of claim 7, wherein the recommendation workflow includes a matching process implemented using one or more machine learning models, each of the one or more machine learning models generating scores based at least in part on outcomes tracked within patient records of other recovery users.

9. The platform of claim 8, wherein at least one of the one or more machine learning models generates recommendations for candidate peer recovery specialists based on correlations among recovery user outcomes obtained from patient records and characteristics of candidate peer recovery specialists.

10. The platform of claim 1, further including a predictive framework including one or more predictive models usable to determine, based at least in part on patient and outcome data of other recovery users and transaction records of the recovery user, a likelihood of relapse of the recovery user.

11. The platform of claim 10, wherein the predictive framework is configured to generate one or more notifications, the one or more notifications including at least one of a notification to the recovery user identifying a likelihood of relapse and a notification to another user recommending communication with the recovery user.

12. The platform of claim 9, wherein the one or more machine learning models are trained based on both patient and outcome data and a plurality of third party records, and wherein the one or more machine learning models is configured to identify one or more non-users likely to have a substance abuse disorder, and to generate at least one notification to the one or more non-users.

13. A method of managing a substance abuse recovery process for a recovery user at a centralized recovery platform, the method comprising:
receiving a registration of a recovery user in a program managed using the centralized recovery platform;
establishing a relationship with a peer recovery specialist via the centralized recovery platform;
tracking progress of the recovery user through treatment programs and concurrently monitoring status of the recovery user in one or more third party monitoring systems, the status including at least one of government status and employment status;
during the progress of the recovery user through the treatment programs, providing the recovery user with information regarding qualification as a peer recovery specialist; and
upon meeting predetermined recovery criteria and training criteria, qualifying the recovery user as a peer recovery specialist at the centralized recovery platform.

14. The method of claim 13, further comprising associating one or more subsequent recovery users with the recovery user, wherein the recovery user corresponds to a peer recovery specialist with respect to the one or more subsequent recovery users.

15. The method of claim 13, further comprising generating one or more certification reports for the user from the centralized recovery platform, the one or more certification reports indicating a validated training status or recovery program completion status of the recovery user.

16. The method of claim 13, wherein the government status corresponds to a court or probation status of the recovery user, and wherein the employment status corresponds to a current or maintained employment status of the recovery user during a treatment period.

17. The method of claim 13, further comprising presenting, in a user interface, one or more recommended peer recovery specialists to the recovery user based on historical outcome information from patient records of other recovery users.

18. The method of claim 17, further comprising presenting, in a user interface, one or more recommended services to the recovery user based on the historical outcome information.

19. The method of claim 18, wherein the historical outcome information includes historical treatment success outcomes of other recovery users and information regarding peer recovery specialists and services used by the other recovery users as reflected in the patient records of the other recovery users.

20. The method of claim 19, wherein the one or more recommended peer recovery specialists includes available peer recovery specialists.
